(19) United States
(12) Patent Application Publication
(10) Pub. No.: US 2014/0296893 A1
(43) Pub. Date: Oct. 2, 2014

(54) LIQUID EJECTION DEVICE AND MEDICAL APPARATUS

(71) Applicant: SEIKO EPSON CORPORATION, Tokyo (JP)

(72) Inventors: Hideki KOJIMA, Matsumoto-shi (JP); Hirokazu SEKINO, Chino-shi (JP)

(73) Assignee: SEIKO EPSON CORPORATION, Tokyo (JP)

(21) Appl. No.: 14/227,136

(22) Filed: Mar. 27, 2014

(30) Foreign Application Priority Data
Mar. 28, 2013 (JP) 2013-067730

(51) Int. Cl. A61B 17/3203

(52) U.S. Cl. CPC A61B 17/3203; A61B 2017/32032; A61B 2560/02
USPC 606/167; 239/99

(57) ABSTRACT

A liquid ejection device ejecting a liquid in a pulsed manner, includes: an ejection channel that includes an ejection port ejecting the liquid; an actuator that transports pulsed flow to the ejection channel by pressurizing the liquid supplied from outside; a suction channel that includes a suction port capable of sucking the liquid; and a storage container that accommodates a part of the suction channel and the actuator, and in which the ejection channel extends from one end thereof, in which the ejection channel is disposed near the suction channel on a side of the ejection port and is separated from the suction channel by being curved in the storage container, and in which the suction channel is a linear shape or a curved shape having a curvature that is less than that of a curve of the ejection channel in a portion in which the ejection channel is separated.

This application claims the benefit of Japanese Patent Application No. 2013-67730, filed on Mar. 28, 2013. The content of the aforementioned application is incorporated herein by reference in its entirety.

### BACKGROUND

1. Technical Field

The present invention relates to technology of a liquid ejection device that ejects a liquid and a medical apparatus that uses the liquid ejection device.

2. Related Art

For example, technology regarding a liquid ejection device that ejects a liquid is disclosed in JP-A-2011-177407. The liquid ejection device disclosed in JP-A-2011-177407 describes technology in which excised matter that is excised by a liquid that is ejected from an ejection channel is removed by sucking in a suction channel.

The liquid ejection device described in JP-A-2011-177407 has an excellent structure in that a suction tube is externally inserted with respect to the ejection channel that is led out linearly from an actuator pressing the liquid and a pressurized fluid that is pressurized by the actuator is guided to an ejection port. Further, since a suction port of the suction tube is positioned immediately near the ejection port, it is excellent in that the excised matter excised by the liquid that is ejected can be efficiently sucked.

A structure is pursued in which clogging or the like of the suction channel is unlikely to occur by reducing a channel resistance of the suction channel while taking advantage of such a structure. Further, in the liquid ejection device, small size, low cost, resource saving, ease of manufacturing or improvement of usability is desired.

### SUMMARY

An advantage of some aspect of the invention is to solve at least a part of the problems described above, and the invention can be implemented as the following aspects.

(1) An aspect of the invention provides a liquid ejection device. The liquid ejection device includes: an ejection channel that includes an ejection port ejecting the liquid; an actuator that transports pulsed flow to the ejection channel by pressurizing the liquid supplied from outside; a suction channel that includes a suction port capable of sucking the liquid; and a storage container that accommodates a part of the suction channel and the actuator, and in which the ejection channel extends from one end thereof. The ejection channel is disposed near the suction channel on a side of the ejection port and is separated from the suction channel by being curved in the storage container. The suction channel is a linear shape or a curved shape having a curvature that is less than that of a curve of the ejection channel in a portion in which the ejection channel is separated. In this case, since the ejection channel is curved and then is separated from the suction channel, it is possible to suppress clogging on the inside of the suction channel due to the suction matter and to smoothly perform the suction compared to in a case where the suction channel is separated with respect to the ejection channel by being curved. Further, since the suction channel is the linear shape or the curved shape having a curvature that is less than that of the ejection channel in the portion in which the ejection channel is separated, it is possible to suppress the clogging of the suction matter that is sucked. Further, the ejection channel is separated by being curved, but the ejection channel may be curved as long as the ejection channel can propagate the pressure of the liquid. Further, a field of view cannot be interfered with a finger or the like at the time of grasping by bending the ejection channel to be curved.

(2) In the liquid ejection device of the aspect described above, the storage container maybe a columnar shape; the ejection channel may be configured to include a first channel that is integrally disposed with the suction channel and a second channel that is separated from the suction channel; and the second channel after being separated may be disposed in a long axial direction of the storage container or substantially parallel to the suction channel in the storage container. According to this aspect, it is possible to suppress enlarging of a diameter of a cross section perpendicular to the long axial direction of the storage container. Further, since when the ejection channel and the suction channel are led out from the storage container to the outside, they have the same direction as each other, the ejection channel and the suction channel are easily arranged.

(3) In the liquid ejection device of the aspect described above, the suction channel may be led out from a leading end of the storage container to the outside by inserting the ejection channel into the suction channel on the inside of the storage container, and a channel in which the sucked liquid flows maybe formed between an outer wall of the ejection channel and an inner wall of the suction channel. According to this aspect, it is possible to reduce a combined diameter of the ejection channel and the suction channel which are led out from the storage container.

(4) In the liquid ejection device of the aspect described above, the suction channel may have a suction adjustment hole that adjusts a degree of the suction, and the suction adjustment hole maybe provided on the suction channel after the ejection channel is separated. According to this aspect, it is possible to suppress influence of change in the pressure inside the suction channel with the opening and closing of the suction adjustment hole to the ejection channel, and to stably eject the pulsed flow. Further, since the suction channel is the linear shape or the curved shape having the curvature that is less than that of the curve of the ejection channel, a user easily and sensorily finds a positional relationship between a position in which the suction is operated and the suction adjustment hole to be operated, thereby improving usability.

(5) Another aspect of the invention provides a medical apparatus using the liquid ejection device of the aspect described above. According to this aspect, it is possible to suppress the clogging of the suction channel due to the suction matter.

Not all of a plurality of configuration elements included in each aspect of the invention described above are essential. A configuration element that is a part of the plurality of configuration elements described above may be appropriately changed, deleted, and replaced with other, new configuration elements, and partial deletion of a limited content may be performed. Further, an independent aspect of the invention may be achieved by combining a part of or all of technical characteristics that are included in one aspect of the invention described above with a part of or all of technical characteristics that are included in another aspect of the invention described above.

For example, an aspect of the invention may be implemented as a device that includes one or more elements among four elements of the ejection channel, the actuator, the storage container and the suction channel. That is, the device may have or may not have the ejection channel. Further, the device may have or may not have the actuator. Further, the device may have or may not have the storage container. Further, the device may have or may not have the suction channel. For example, the ejection channel maybe configured as an ejection channel having an ejection port that ejects a liquid. The actuator may be configured as an actuator that transports pulsed flow to the ejection channel by pressurizing the liquid supplied from the outside. For example, the storage container may be configured as a storage container that includes the ejection channel in one end thereof and stores the actuator on the inside thereof. For example, the suction channel may be configured as a suction channel that includes a suction port capable of sucking the liquid that is ejected. For example, such a device can be implemented as the liquid ejection device and can be also implemented as another device other than the liquid ejection device. According to the aspect described above, it is possible to achieve at least one of small size, low cost, resource saving, ease of manufacturing, improvement of usability and the like. A part or all of the technical characteristics of each aspect of the liquid ejection device described above may be applied to the device.

The invention can be implemented in various aspects other than the device. For example, the invention can be implemented in an aspect of a method for ejecting the liquid or a method for manufacturing the liquid ejection device.

### BRIEF DESCRIPTION OF THE DRAWINGS

The invention will be described with reference to the accompanying drawings, wherein like numbers reference like elements.

FIG. 1 is an explanatory view describing a configuration of a liquid ejection device.

FIG. 2 is an explanatory view describing an internal structure of a handpiece.

FIG. 3 is an explanatory view illustrating a handpiece as Modification Example 1.

### DESCRIPTION OF EXEMPLARY EMBODIMENTS

### A. FIRST EMBODIMENT

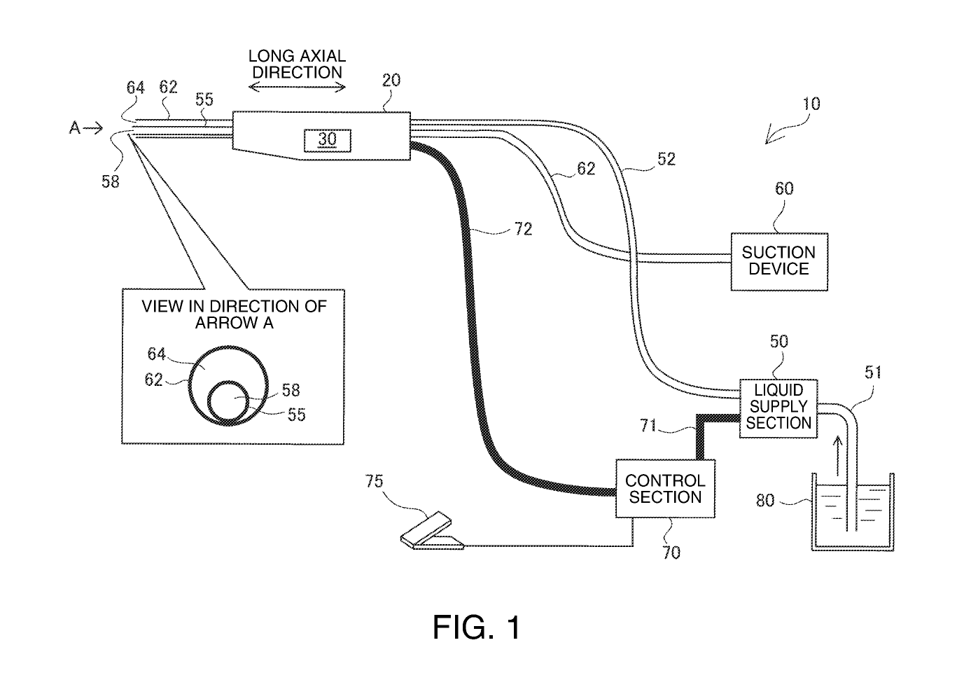

FIG. 1 is an explanatory view describing a configuration of a liquid ejection device 10 as a first embodiment of the invention. The liquid ejection device 10 of the embodiment is a medical apparatus used in medical institutions and has a function as a scalpel for performing incision on or excision of a diseased part by ejecting the liquid with respect to the diseased part.

The liquid ejection device 10 includes a handpiece 20 as a storage container, a liquid supply section 50, a suction device 60 and a control section 70, and is connected to a liquid container 80. The liquid supply section 50 and the liquid container 80 are connected by a connection tube 51. The liquid supply section 50 and the handpiece 20 are connected by a liquid supply channel 52. In the embodiment, the connection tube 51 and the liquid supply channel 52 are formed by a resin.

The liquid container 80 accommodates physiological saline solution as the liquid. The liquid supply section 50 supplies the liquid that is sucked from the liquid container 80 through the connection tube 51 to the handpiece 20 through the liquid supply channel 52. Moreover, in the embodiment, the physiological saline solution is employed as the liquid, but the invention is not limited to the embodiment and it is possible to employ various types of liquids such as sterile water or pure water.

The handpiece 20 is a device that is grasped and operated by a user of the liquid ejection device 10. The handpiece 20 includes an actuator 30, an ejection tube 55, an ejection port 58, and a suction force adjustment mechanism 65. A direction along the ejection tube 55 is referred to as a long axial direction. Pulsation having a predetermined frequency is applied to the liquid, which is supplied from the liquid supply section 50 to the handpiece 20 through the liquid supply channel 52, by the actuator 30, and the liquid is supplied to the ejection tube 55. The liquid supplied to the ejection tube 55 is ejected from the ejection port 58 as a pulsed liquid. The user performs incision on or excision of a diseased part by applying the pulsed liquid ejected from the ejection port 58 to the diseased part of the patient. In the embodiment, the ejection tube 55 is formed of stainless steel. However, the ejection tube 55 may be formed of a material having a predetermined rigidity or higher such as other metals, for example, brass or the like, or reinforced plastic. The suction force adjustment mechanism 65 is a mechanism capable of adjusting a degree of the suction of the suction device 60 by the user. In the embodiment, as the degree of the suction, a suction amount per unit time is employed.

The control section 70 transmits a drive signal to the actuator 30 through a signal cable 72 and controls the liquid supply section 50 through a control cable 71, thereby performing control of a flow amount of the liquid that is supplied to the actuator 30. A foot switch 75 that is operated by a foot of the user is connected to the control section 70. When the user turns on the foot switch 75, the control section 70 controls the liquid supply section 50 and performs the supply of the liquid to the actuator 30, and transmits the drive signal to the actuator 30. Therefore, the pulsation is applied to the liquid that is supplied to the actuator 30 and the pulsed liquid is ejected from the ejection port 58. Moreover, the expression “the liquid is ejected in a pulsed manner” means that the liquid is ejected in a state where changes in the flow amount or a flow speed of the liquid that is ejected are accompanied. Intermittent ejection in which the liquid is ejected by repeating the ejection and stop of the liquid is included in the form in which the liquid is ejected in the pulsed manner. However, as long as the flow amount or the flow speed of the liquid changes, it is not necessary for the form to be the intermittent ejection.

The suction device 60 is a device that sucks the liquid, excised matter or the like (hereinafter, also referred to as suction matter) around the ejection port 58. The suction device 60 and the handpiece 20 are connected by a suction channel 62. The suction channel 62 extends near a leading end of the ejection tube 55 through inside the handpiece 20. The ejection tube 55 is inserted into the inside of the suction channel 62. As illustrated in a direction of arrow A in FIG. 1, a channel (hereinafter, also referred to as a gap channel) in which the liquid, which is sucked from a suction port 64 that is the leading end of the suction channel 62, flows, is formed between an outer wall of the ejection tube 55 and an inner wall of the suction channel 62. The liquid that flows from the suction port 64 into the gap channel is sucked into the suction device 60 through a suction channel 62. The suction amount per unit time that is sucked from the suction port 64 is capable of being adjusted by operating the suction force adjustment mechanism 65 by the user.

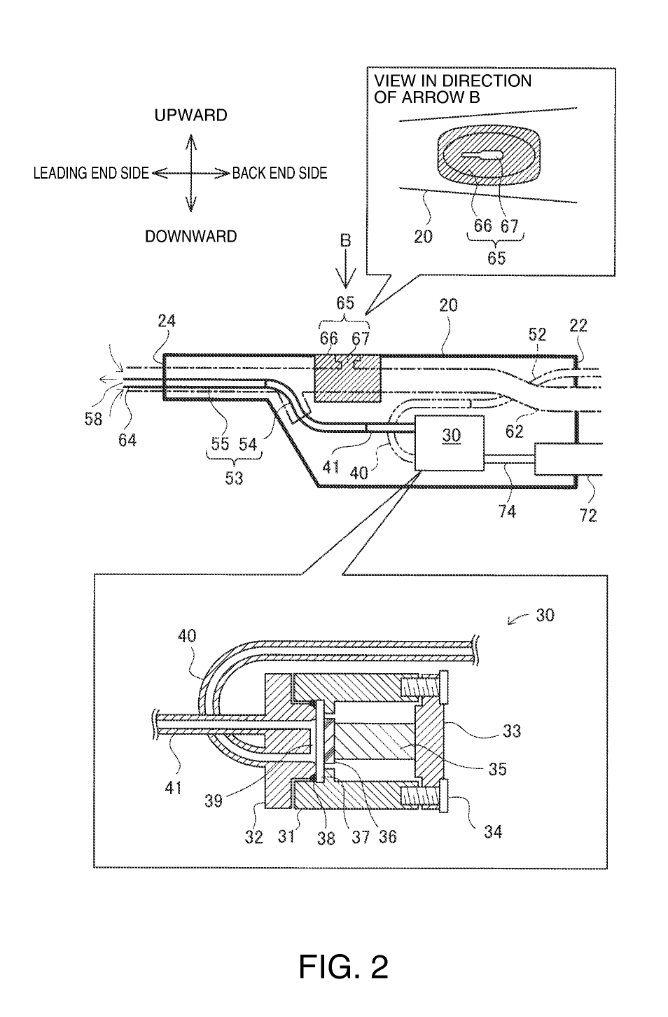

FIG. 2 is an explanatory view describing an internal structure of the handpiece 20. As described above, the actuator 30 is accommodated inside the handpiece 20. As illustrated in a lower section of the view, the actuator 30 includes a first case 31, a second case 32, a third case 33, a bolt 34, a piezoelectric element 35, a reinforced plate 36, a diaphragm 37, a gasket 38, an inlet channel 40, and an outlet channel 41. The first case 31 and the second case 32 are bonded by facing each other. The first case 31 is a cylindrical member. One end section of the first case 31 is closed by fixing the third case 33 with the bolts 34. The piezoelectric element 35 is disposed in a space that is formed inside the first case 31.

The piezoelectric element 35 is a multilayered piezoelectric element. One end section of the piezoelectric element 35 is fixed to the diaphragm 37 through the reinforced plate 36. The other end section of the piezoelectric element 35 is fixed to the third case 33. The diaphragm 37 is formed of a metal thin film and a peripheral section thereof is fixed to the first case 31. A liquid chamber 39 is formed between the diaphragm 37 and the second case 32. The volume of the liquid chamber 39 is changed by driving of the piezoelectric element 35.

The inlet channel 40 into which the liquid flows and the outlet channel 41 from which the liquid flows are formed in the second case 32. The inlet channel 40 extends from a position deviated from a center of an end surface of the second case 32 and is bent upward in a U-shape in the view, and stretches to a back end section 22 of the handpiece 20. As described above, it is possible to reduce a curvature of the U-shape portion of the inlet channel 40 by bending the inlet channel 40. Further, it is possible to reduce a combined width of the inlet channel 40 stretching to the back end section 22 and the actuator 30, and as a result, it is possible for the handpiece 20 that accommodates the actuator 30 and the inlet channel 40 to be thin. Further, it is possible to position the center of gravity of the handpiece 20 on the side of a leading end section 24 by stretching the inlet channel 40 from the side of the leading end section 24 of the actuator 30.

The liquid supply channel 52 is connected to the inlet channel 40. The ejection tube 55 is connected to the outlet channel 41 through a connection tube 54. A channel that is formed of two tube paths of the connection tube 54 and the ejection tube 55 is also referred to as an ejection channel 53. The liquid supplied from the liquid supply section 50 is supplied to the liquid chamber 39 through the liquid supply channel 52. When the piezoelectric element 35 vibrates with a predetermined frequency, the volume of the liquid chamber 39 is changed through the diaphragm 37 and the liquid that is accommodated is pressurized. The liquid that is pressurized is discharged from the ejection port 58 through the outlet channel 41, the connection tube 54 and the ejection tube 55.

The suction channel 62 communicates with the leading end section 24 from the back end section 22 of the handpiece 20. On the inside of the handpiece 20, the ejection channel 53 is inserted into the suction channel 62. The suction channel 62 is led out from the leading end section 24 of the handpiece 20 together with the ejection tube 55.

The ejection channel 53 and the suction channel 62 are separated on the inside of the handpiece 20. The ejection channel 53 is separated from the suction channel 62 by being curved in a separation portion of two channels. The ejection channel 53 is gently curved and the curvature is preferably decreased in the separation portion. The suction channel 62 is arranged substantially linearly in the separation portion. Moreover, in the embodiment, an aspect in which the suction channel 62 is arranged substantially linearly in the separation portion is employed, but the suction channel 62 maybe disposed in a curved shape having the curvature less than that of the curve of the ejection channel in the separation portion.

The connection tube 54 among the tube paths configuring the ejection channel 53 is disposed substantially parallel to the long axial direction of the handpiece 20 that is a columnar shape and is connected to the outlet channel 41 after being separated from the suction channel 62. Further, in the embodiment, the connection tube 54 after being separated is disposed substantially parallel to the suction channel 62.

The suction force that sucks the liquid or the like from the suction port 64 by the suction channel 62 is capable of being adjusted in the suction device 60 and is capable of being adjusted by operating an operation section 66 of the suction force adjustment mechanism 65 included in the handpiece 20 by the user.

The suction force adjustment mechanism 65 includes the operation section 66 and a suction adjustment hole 67. The suction force adjustment mechanism 65 is a member that is formed of a resin. A channel configuring a part of the suction channel 62 is formed inside the suction force adjustment mechanism 65. Practically, the suction channel 62 is connected to both ends of the channel formed in the suction force adjustment mechanism 65. The operation section 66 is a portion among the suction force adjustment mechanism 65 which is exposed to the outside of the handpiece 20 and is a portion that is operated with a finger of the user.

The suction adjustment hole 67 communicates with the suction channel 62 and the operation section 66. As illustrated in a direction of arrow B in FIG. 2, an opening section of the suction adjustment hole 67 is formed in the operation section 66. The user opens and closes the suction adjustment hole 67 with a finger when grasping the handpiece 20. The suction force adjustment mechanism 65 adjusts the flow amount of air flowing from the outside into the suction channel 62 through the suction adjustment hole 67 and adjusts the pressure (hereinafter, also referred to as a suction pressure) inside the suction channel 62, depending on a size of a surface area of the suction adjustment hole 67 that is closed by the user. That is, the suction force adjustment mechanism 65 adjusts the suction amount per unit time.

The suction adjustment hole 67 is provided on the suction channel 62 after the ejection tube 55 is separated. In other words, the suction adjustment hole 67 is provided on the suction channel 62 on the back end side from the separation portion of the ejection tube 55 and the suction channel 62.

If the suction force adjustment mechanism 65 is directed upward with respect to gravity, the suction matter is difficult to be sucked from the suction adjustment hole 67 to the outside and then the function thereof is preferably exerted. When grasping the handpiece 20, the user adjusts the suction amount per unit time of the suction device 60 by directing the suction force adjustment mechanism 65 upward and by pressing the suction adjustment hole 67 included in the operation section 66 downward with a finger. Moreover, when grasping the suction force adjustment mechanism 65 in the upward direction with respect to the gravity, a position of each configuration of the liquid ejection device 10 is determined so that the function of the suction force adjustment mechanism 65 or operability of the user is suitable, but the suction force adjustment mechanism 65 does not force the user to necessarily use the handpiece 20 in the upward direction.

The signal cable 72 is inserted from the back end section 22 of the handpiece 20. Two electrode wires 74 consisting of plus and minus inserted into the signal cable 72 are connected to the piezoelectric element 35 inside the actuator 30. The drive signal transmitted from the control section 70 is transmitted to the piezoelectric element 35 through the electrode wires 74 inside the signal cable 72. The piezoelectric element 35 expands and contracts, based on the drive signal.

As described above, since the ejection channel 53 is curved and separated, it is possible to suppress clogging the inside of the suction channel 62 by the suction matter compared to in a case where the suction channel 62 is separated with respect to the ejection channel 53 by being curved. Since the suction channel 62 is substantially linearly disposed in the portion in which the ejection channel 53 is separated, it is possible to reduce a channel resistance and to suppress clogging by the suction matter that is sucked. Further, the ejection channel 53 is separated with respect to the suction channel 62 by being curved, but the ejection channel 53 may be curved as long as the pressure of the liquid can be propagated. The ejection channel 53 is shifted with respect to the center shaft of the handpiece 20 in the long axial direction and a field of view is unlikely to be interfered with a finger or the like of the user by bending the ejection channel 53 to be curved with respect to the suction channel 62.

The connection tube 54 configuring the ejection channel 53 is disposed substantially parallel in the long axial direction of the handpiece 20 that is a columnar shape after being separated from the suction channel 62. Therefore, it is possible to avoid enlarging of a diameter of a cross section perpendicular to the long axial direction of the handpiece 20 to accommodate the suction channel 62 inside the handpiece 20.

Since the ejection tube 55 is inserted into the suction channel 62 and then the suction channel 62 is led out from the leading end of the handpiece 20 to the outside, it is possible to reduce the diameter of the tube path that is a combination of the suction channel 62 and the ejection tube 55. Thus, the user can ensure a wide field of view when applying the ejection port 58 to the diseased part.

Since the suction adjustment hole 67 is provided on the suction channel 62 after the ejection channel 53 is separated, it is possible to suppress the influence of change in the pressure inside the suction channel 62 due to the opening and closing of the suction adjustment hole 67 to the ejection channel 53. That is, the ejection channel 53 is prevented from being oscillated by the change of the pressure inside the suction channel 62. Therefore, it is possible to suppress the oscillation of the ejection tube 55 or the ejection port 58 and, as a result, it is possible to eject the stable pulsed flow.

In the embodiment, since the separation portion of the suction channel 62 and the ejection channel 53 is formed inside the handpiece 20, it is possible to increase the strength of the separation portion compared to in a case where the separation portion is formed outside the handpiece 20. Since the separation portion is formed inside the handpiece 20, it is possible to avoid the deformation of the separation portion or unnecessary vibration thereof when the user operates the handpiece 20. As a result, it is possible to eject the stable pulsed flow. Further, it is possible to stabilize the suction pressure inside the suction channel 62.

As a corresponding relationship between the embodiment described above and the appended claims, the handpiece 20 corresponds to a storage container described in the appended claims, the ejection tube 55 corresponds to a first channel described in the appended claims, and the connection tube 54 corresponds to a second channel described in the appended claims.

### B. MODIFICATION EXAMPLE

Moreover, the invention is not limited to the embodiment or embodiments described above and it is possible to implement the invention in various embodiments within the scope that does not depart from the spirit thereof and, for example, the following variations are possible.

### B1 Modification Example 1

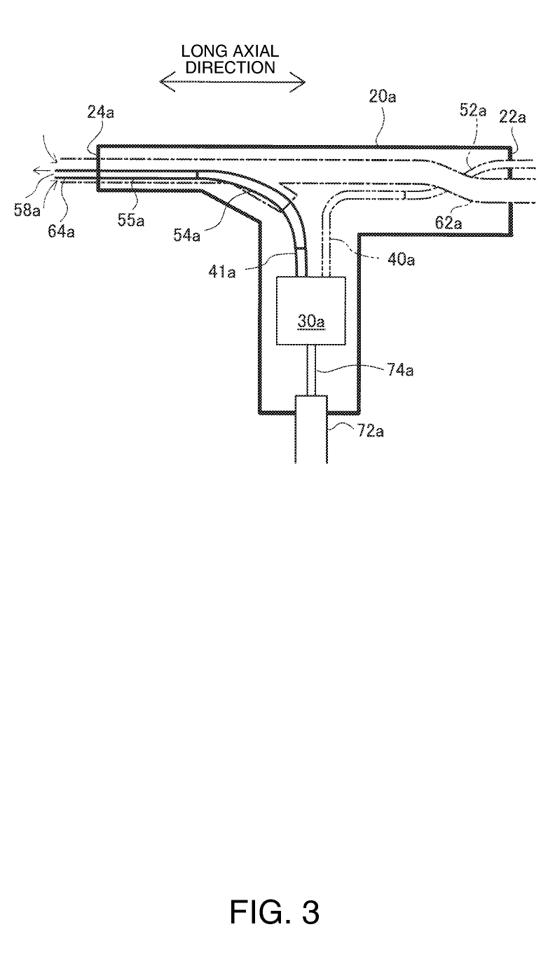

In the embodiment described above, the connection tube 54 is disposed substantially parallel in the long axial direction of the handpiece 20 having the columnar shape after being separated from the suction channel 62, but the invention is not limited to the embodiment and another mode may be employed. FIG. 3 is an explanatory view illustrating an example of a handpiece 20a as Modification Example 1. As described in the view, a connection tube 54a may be disposed in a direction intersecting a long axial direction of the handpiece 20a after being separated from a suction channel 62a. Also in this case, since an ejection tube 55a is separated from the suction channel 62a by being curved, it is possible to suppress clogging the inside of the suction channel 62a by the suction matter compared to in a case where the suction channel 62a is separated with respect to the ejection tube 55a by being curved. Since the suction channel 62a is substantially linearly disposed in a portion in which the ejection tube 55a is separated, it is possible to reduce a channel resistance and to suppress clogging by the suction matter that is sucked. Further, the ejection tube 55a is separated with respect to the suction channel 62a by being curved, but the ejection tube 55a may be curved as long as the pressure of the liquid is propagated.

### B2 Modification Example 2

In the embodiment described above, the liquid ejection device 10 includes the suction force adjustment mechanism 65 (the operation section 66 and the suction adjustment hole 67), but the invention may not include the suction force adjustment mechanism 65. Also in this case, it is possible to obtain the same advantage as that of the embodiment described above.

### B3 Modification Example 3

In the embodiment described above, the aspect is employed in which the suction channel 62 is disposed substantially linearly in the separation portion of the ejection channel 53 and the suction channel 62, but the suction channel 62 may be disposed in a curved shape having a curvature less than that of the curve of the ejection channel 53 in the separation portion. Also in this case, it is possible to obtain the same advantage as that of the embodiment described above.

### B4 Modification Example 4

In the embodiment described above, the liquid ejection device 10 is used as the medical apparatus. However, the liquid ejection device 10 may be used as an apparatus other than the medical apparatus. For example, the liquid ejection device 10 may be used as a cleaning apparatus for removing soil of an object by applying the liquid that is ejected to the object, or a depiction apparatus that draws characters, graphics or the like by the liquid that is ejected. Also in this case, it is possible to obtain the same advantage as that of the embodiment described above.

### B5 Modification Example 5

In the embodiment described above, the physiological saline solution is employed as the liquid, but the invention is not limited to the embodiment and it is possible to employ various types of liquids such as sterile water or ultrapure water.

### B6 Modification Example 6

In the embodiment described above, the suction amount per unit time is used as the degree of the suction that is adjusted by the suction force adjustment mechanism, but the invention is not limited to the embodiment and, for example, various types of physical quantities capable of defining the degree of the suction such as using the suction force are employed and the physical quantities may be adjusted.

### B7 Modification Example 7

In the embodiment described above, the aspect is employed in which the ejection tube 55 is inserted into the suction channel 62 as a disposition relationship of the ejection tube 55 and the suction channel 62 which are led out from the leading end section 24 of the handpiece 20, but the invention is not limited to the embodiment. For example, it is possible to employ various aspects such as employing an aspect that the outer wall of the ejection tube 55 and the outer wall of the suction channel 62 are led out from the leading end section 24 by being adjacent to each other. In order to suck the liquid around the ejection port 58 from the suction port 64, it is preferable that the ejection tube 55 and the suction channel 62 be integrally led out from the leading end section 24 as in the embodiment described above and in this Modification Example.

What is claimed is:

1. A liquid ejection device ejecting a liquid in a pulsed manner, comprising:
an ejection channel that includes an ejection port ejecting the liquid;
an actuator that transports pulsed flow to the ejection channel by pressurizing the liquid supplied from outside;
a suction channel that includes a suction port capable of sucking the liquid; and
a storage container that accommodates a part of the suction channel and the actuator, and in which the ejection channel extends from one end thereof,
wherein the ejection channel is disposed near the suction channel on a side of the ejection port and is separated from the suction channel by being curved in the storage container, and
wherein the suction channel is a linear shape or a curved shape having a curvature that is less than that of a curve of the ejection channel in a portion in which the ejection channel is separated.

2. The liquid ejection device according to claim 1,
wherein the ejection channel is configured to include a first channel that is integrally disposed with the suction channel and a second channel that is separated from the suction channel, and
wherein the second channel after being separated is disposed in a long axial direction of the storage container or substantially parallel to the suction channel in the storage container.

3. The liquid ejection device according to claim 1,
wherein the suction channel is led out from a leading end of the storage container to the outside by inserting the ejection channel into the suction channel on the inside of the storage container, and a channel in which the sucked liquid flows is formed between an outer wall of the ejection channel and an inner wall of the suction channel.

4. The liquid ejection device according to claim 1,
wherein the suction channel has a suction adjustment hole that adjusts a degree of the suction, and
wherein the suction adjustment hole is provided on the suction channel after the ejection channel is separated.

5. A medical apparatus using the liquid ejection device according to claim 1.

6. A medical apparatus using the liquid ejection device according to claim 2.

7. A medical apparatus using the liquid ejection device according to claim 3.

8. A medical apparatus using the liquid ejection device according to claim 4.
